(19) United States
(12) Patent Application Publication
(10) Pub. No.: US 2025/0166193 A1
(43) Pub. Date: May 22, 2025

(54) MEDICAL IMAGING ANNOTATION DEVICE AND MEDICAL IMAGING ANNOTATION METHOD

(71) Applicant: HTC Corporation, Taoyuan City (TW)

(72) Inventors: Chih-Jou HSU, Taoyuan City (TW); Yu-Shao PENG, Taoyuan City (TW)

(21) Appl. No.: 18/948,555

(22) Filed: Nov. 15, 2024

Related U.S. Application Data
(60) Provisional application No. 63/599,565, filed on Nov. 16, 2023.

(51) Int. Cl. G06T 7/00

(52) U.S. Cl. CPC G06T 7/0014; G06T 2207/10081

(57) ABSTRACT

A medical imaging annotation device includes an interface, a memory and a processor. The memory is configured to store serialized medical images. The processor is coupled to the interface and the memory. The processor is configured to receive, via the interface, a first manual annotation on a first slice image of the serialized medical images and a second manual annotation on a second slice image of the serialized medical images. The processor is configured to execute a bidirectional inference mechanism to generate final annotation labels on intermediate slice images of the serialized medical images based on the first manual annotation and the second manual annotation respectively.

### RELATED APPLICATIONS

This application claims the priority benefit of U.S. Provisional Application Ser. No. 63/599,565, filed Nov. 16, 2023, which is herein incorporated by reference.

### BACKGROUND

### Field of Invention

The present disclosure relates to a medical imaging technology. More particularly, the disclosure relates to methods and devices for annotating serialized medical images.

### Description of Related Art

Medical imaging technologies, including X-rays, Magnetic Resonance Imaging (MRI), and Computed Tomography (CT), are critical tools in modern diagnostics, providing detailed internal structural images necessary for accurate diagnosis and treatment planning. However, the increasing volume and complexity of imaging data pose significant challenges for healthcare professionals, particularly in the precise identification and annotation of Regions of Interest (RoI).

However, annotating RoIs in a large amount of medical imaging is highly time-consuming. In some practices, accurately annotating Regions of Interest (RoI) often relied on manual operations by radiologists or radiographic technicians. This method is particularly burdensome when dealing with serialized imaging such as CT and MRI, as it requires professionals to annotate each slice obtained from the scan individually. This process not only consumes a significant amount of time but also introduces inconsistencies due to variances among annotators, which can affect the efficiency of medical image analysis and clinical diagnosis.

### SUMMARY

The disclosure provides a medical imaging annotation device, which includes an interface, a memory and a processor. The memory is configured to store serialized medical images. The processor is coupled to the interface and the memory. The processor is configured to receive, via the interface, a first manual annotation on a first slice image of the serialized medical images and a second manual annotation on a second slice image of the serialized medical images. The processor is configured to execute a bidirectional inference mechanism to generate final annotation labels on intermediate slice images of the serialized medical images based on the first manual annotation and the second manual annotation respectively.

The disclosure also provides a medical imaging annotation method, which includes steps of: obtaining the serialized medical images; receiving a first manual annotation on a first slice image of the serialized medical images and a second manual annotation on a second slice image of the serialized medical images; executing a bidirectional inference mechanism to generate final annotation labels on intermediate slice images of the serialized medical images based on the first manual annotation and the second manual annotation respectively.

It is to be understood that both the foregoing general description and the following detailed description are demonstrated by examples, and are intended to provide further explanation of the invention as claimed.

### BRIEF DESCRIPTION OF THE DRAWINGS

The disclosure can be more fully understood by reading the following detailed description of the embodiment, with reference made to the accompanying drawings as follows:

FIG. 1 is a schematic diagram illustrating a medical imaging annotation device according to an embodiment of the disclosure.

FIG. 2 is a schematic diagram illustrating a first manner to implement the bidirectional inference mechanism executed by the processor according to an embodiment of the disclosure.

FIG. 3 is a schematic diagram illustrating how the tracking model generates the forward prediction annotations.

FIG. 4 is a schematic diagram illustrating how the tracking model generates the backward prediction annotations.

FIG. 5 is a schematic diagram illustrating a bidirectional XMem model executed by the processor according to an embodiment of the disclosure.

FIG. 6 is a schematic diagram illustrating the bidirectional XMem model executed by the processor regarding to another pair of slice images.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

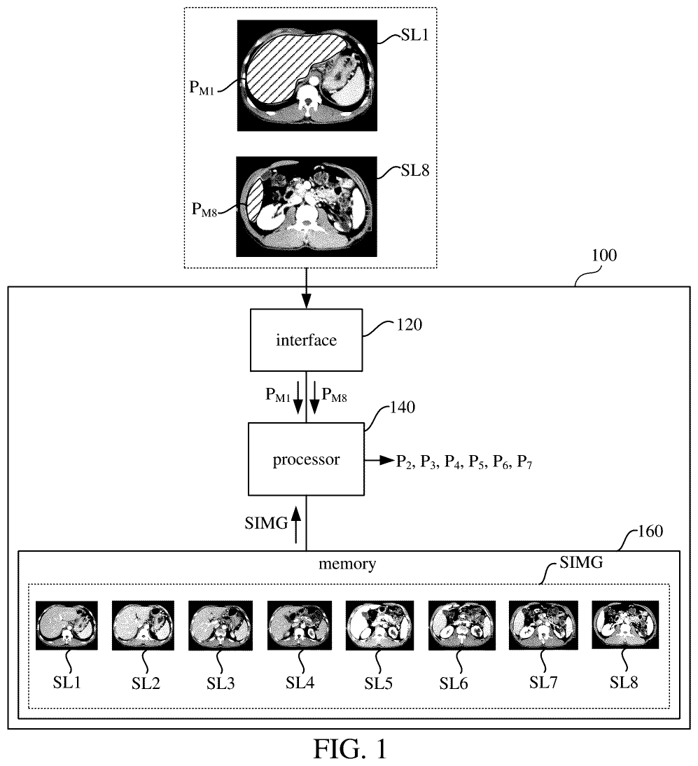

Reference is made to FIG. 1. FIG. 1 is a schematic diagram illustrating a medical imaging annotation device 100 according to an embodiment of the disclosure. In some embodiments, the medical imaging annotation device 100 is configured to generate annotations on serialized medical images SIMG in an efficient way.

In some embodiments, the serialized medical images SIMG include Magnetic Resonance Imaging (MRI) scan images or Computed Tomography (CT) scan images. As embodiments shown in FIG. 1, the serialized medical images SIMG can be a series of abdominal MRI/CT scan images. The serialized medical images SIMG may include multiple slice images captured in sequence. As shown in FIG. 1, the serialized medical images SIMG includes slice images SL1, SL2, SL3, SL4, SL5, SL6, SL7 and SL8. In some embodiments, these slice images SL1 to SL8 can be captured from a patient by a MRI/CT scanner at different timings. For example, firstly, the slice image SL1 can be captured; the slice image SL2 can be captured after the slice image SL1; the slice image SL3 can be captured after the slice image SL2, and so on. The slice image SL8 can be captured at last. These slice images SL1 to SL8 can represent sequential abdominal images at different heights.

The serialized medical images SIMG are not limited to the abdominal MRI/CT scan images shown in FIG. 1. In other embodiments, the serialized medical images SIMG can be a series of brain MRI/CT scan images, a series of chest MRI/CT scan images or other similar medical images. The medical imaging annotation device 100 can be utilized to process various types of the serialized medical images SIMG and have the potential to be extended to other types of medical videos.

For brevity, the serialized medical images SIMG shown in FIG. 1 include total 8 slice images for demonstration. However, an amount of the serialized medical images SIMG in this disclosure is not limited thereto. The serialized medical images SIMG can include N slice images, and N is a positive integer larger than 3.

Because each of the serialized medical images SIMG is captured in sequence, adjacent slice images among the serialized medical images SIMG are relevant to each other. When an object (e.g., an organ or a tissue) appears in the slice image SL1 with a specific contour, the object may also appear in the slice image SL2 with a similar contour. Therefore, if a manual annotation P_{M1} about an object is provided on the slice image SL1 is provided, the manual annotation P_{M1} on the slice image SL1 can be utilized as a hint to segment a similar object in the subsequent slice images SL2, SL3, and so on. Therefore, when another manual annotation P_{M8} about an object is provided on the slice image SL8 is provided, the manual annotation P_{M8} on the slice image SL8 can be utilized as a hint to segment a similar object in the adjacent slice images SL7, SL6, and so on.

The medical imaging annotation device 100 in the disclosure aims to effectively utilize a limited amount of manual-annotated data to enhance the automatic annotation accuracy of algorithms for accurately identifying unlabeled medical images. As shown in FIG. 1, the medical imaging annotation device 100 includes an interface 120, a processor 140 and a memory 160. As shown in FIG. 1, the memory 160 is configured to store the serialized medical images SIMG (i.e., the slice image SL1 to the slice image SL8).

The interface 120 may include Input-Output components (e.g., a touch panel, a keyboard, a mouse, a microphone, a displayer). As shown in FIG. 1, in some embodiments, a user can manipulate the interface 120 to input a manual annotation P_{M1} on the slice image SL1 (ranked at the first slice of the serialized medical images SIMG), and the user can manipulate the interface 120 to input another manual annotation P_{M8} on the slice image SL8 (ranked at the last slice of the serialized medical images SIMG).

As shown in FIG. 1, the processor 140 is coupled with the interface 120 and the memory 160. The processor 140 is configured to receive (via the interface 120) the manual annotation P_{M1} on the slice image SL1 of the serialized medical images SIMG and the manual annotation P_{M8} on the slice image SL8 of the serialized medical images SIMG. The processor 140 is configured to execute a bidirectional inference mechanism to generate final annotation labels P₂, P₃, P₄, P₅, P₆ and P₇ on intermediate slice images (e.g., the slice images SL2, SL3, SL4, SL5, SL6 and SL7) of the serialized medical images SIMG based on the manual annotations P_{M1} and P_{M8}. Details of the bidirectional inference mechanism will be further explained in following embodiments.

In some embodiments, the manual annotations are not limited to be made on the first and the last slice images SL1 and SL8. In other embodiments, the manual annotations can be made on other combinations of the slice images, e.g., on the slice images SL2 and SL7, on the slice images SL3 and SL6.

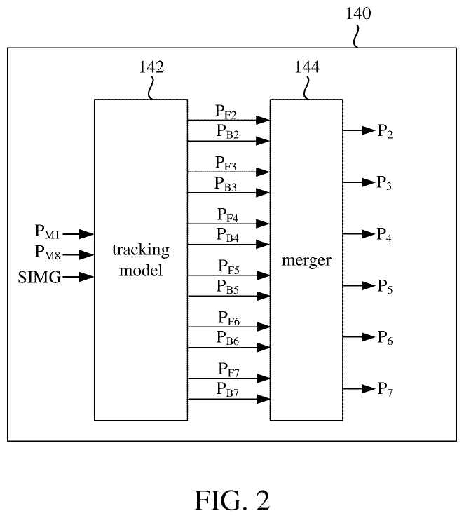

Reference is further made to FIG. 2, which is a schematic diagram illustrating a first manner to implement the bidirectional inference mechanism executed by the processor 140 according to an embodiment of the disclosure. As the embodiment shown in FIG. 2, the bidirectional inference mechanism executed by the processor 140 includes a tracking model 142 and a merger 144. The tracking model 142 is configured to generate forward prediction annotations P_{F2}, P_{F3}, P_{F4}, P_{F5}, P_{F6} and P_{F7} on of the intermediate slice images (e.g., the slice images SL2, SL3 . . . SL7) of the serialized medical images SIMG according to the manual annotation P_{M1} on the slice image SL1. In the meantime, the tracking model 142 is configured to generate backward prediction annotations P_{B7}, P_{B6}, P_{B5}, P_{B4}, P_{B3} and P_{B2} on of the intermediate slice images (e.g., the slice images SL7, SL6 . . . SL2) of the serialized medical images SIMG according to the manual annotation P_{M8} on the slice image SL8.

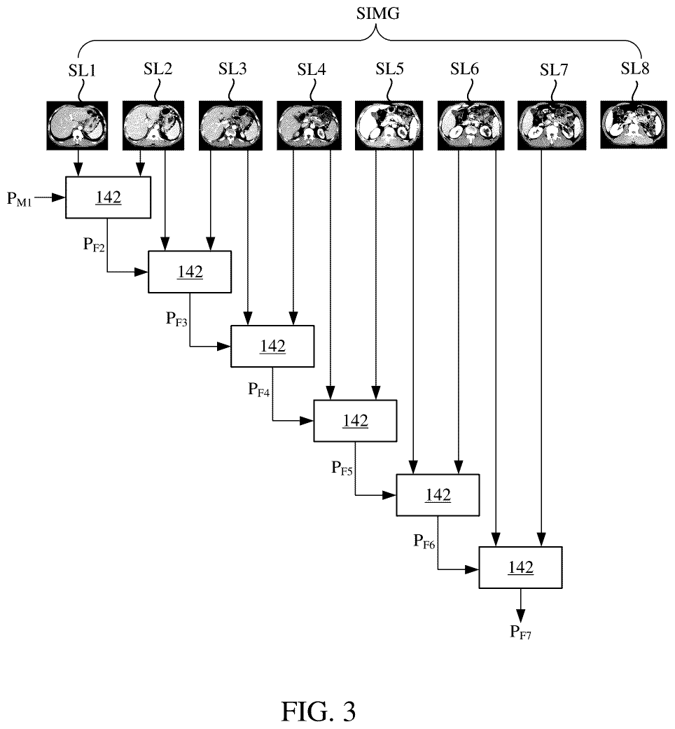

Reference is further made to FIG. 3. FIG. 3 is a schematic diagram illustrating how the tracking model 142 generates the forward prediction annotations P_{F2}, P_{F3}, P_{F4}, P_{F5}, P_{F6} and P_{F7}. In some embodiments, the tracking model 142 can be implemented by a Track-Anything-Model (TAM). Track-Anything-Model (TAM) is configured to detect, associate, and follow the manual annotation P_{M1} on the slice image SL1 across the intermediate slice images (e.g., the slice images SL2, SL3 . . . SL7) in the forward order.

In some embodiment, the Track-Anything-Model is a type of computer vision algorithm designed to follow or track an object or label across the sequence of the serialized medical images SIMG. The Track-Anything-Model can be implemented by software instructions involving convolutional neural networks (CNNs) and recurrent neural networks (RNNs) or transformers. The Track-Anything-Model is pre-trained on vast datasets to understand and predict the motion and appearance of a wide variety of objects.

As shown in FIG. 3, the serialized medical images SIMG and the manual annotation P_{M1} can be provided to the tracking model 142 (e.g., TAM). The tracking model 142 (e.g., TAM) is configured to utilize the manual annotation P_{M1} on the slice image SL1 as a hint to generate the forward prediction annotation P_{F2} about a similar object in the subsequent slice image SL2. In this example, the manual annotation P_{M1} is labeled on a liver area appeared in the slice image SL1, such that the tracking model 142 is able to generate the forward prediction annotation P_{F2} on a potential location of the liver area of the slice image SL2. Similarly, the manual annotation P_{M1} and the forward prediction annotation P_{F2} are utilized by the tracking model 142 as hints to generate the forward prediction annotation P_{F3} about the similar object in the subsequent slice image SL3. In the same manner, the tracking model 142 is able to generate the forward prediction annotations P_{F2}, P_{F3}, P_{F4}, P_{F5}, P_{F6} and P_{F7} on of the intermediate slice images (e.g., the slice images SL2, SL3, SL4, SL5, SL6 and SL7) of the serialized medical images SIMG sequentially in the forward order.

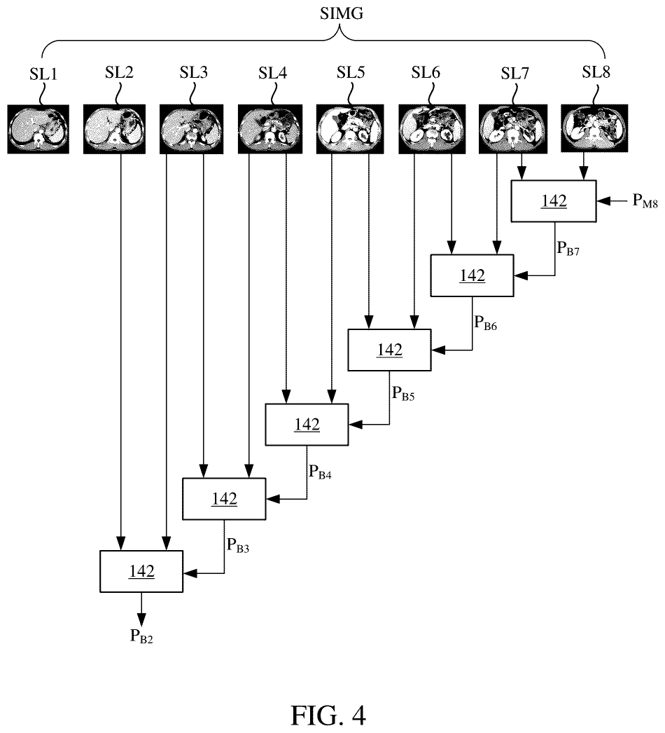

Reference is further made to FIG. 4. FIG. 4 is a schematic diagram illustrating how the tracking model 142 generates the backward prediction annotations P_{B7}, P_{B6}, P_{B5}, P_{B4}, P_{B3} and P_{B2}. In some embodiments, the tracking model 142 can be implemented by the Track-Anything-Model (TAM). Track-Anything-Model (TAM) is configured to detect, associate, and follow the manual annotation P_{M8} on the slice image SL8 across the intermediate slice images (e.g., the slice images SL7, SL6 . . . SL2) in the backward order.

As shown in FIG. 4, the serialized medical images SIMG and the manual annotation P_{M8} can be provided to the tracking model 142 (e.g., TAM). The tracking model 142 (e.g., TAM) is configured to utilize the manual annotation P_{M8} on the slice image SL8 as a hint to generate the backward prediction annotation P_{B7} about a similar object in the adjacent slice image SL7. In this example, the manual annotation P_{M8} is labeled on a liver area appeared in the slice image SL8, such that the tracking model 142 is able to generate the backward prediction annotation P_{B7} on a potential location of the liver area of the slice image SL7. Similarly, the manual annotation P_{M8} and the backward prediction annotation P_{B7} are utilized by the tracking model 142 as hints to generate the backward prediction annotation P_{B6} about the similar object in the adjacent slice image SL6. In the same manner, the tracking model 142 is able to generate the backward prediction annotations P_{B7}, P_{B6}, P_{B5}, P_{B4}, P_{B3} and P_{B2} on of the intermediate slice images (e.g., the slice images SL7, SL6, SL5, SL4, SL3 and SL2) of the serialized medical images SIMG sequentially in the backward order.

As shown in FIG. 2, based on aforementioned bidirectional tracking, the tracking model 142 will generate two prediction annotations for each of the intermediate slice images (e.g., the slice images SL2 to SL7). Regarding to the slice image SL2, there will be one forward prediction annotation P_{F2} (generated based on the manual annotation P_{M1}) and one backward prediction annotation P_{B2} (generated based on the manual annotation P_{M8}). Regarding to the slice image SL3, there will be one forward prediction annotation P_{F3} and one backward prediction annotation P_{B3}. Regarding to the slice image SL7, there will be one forward prediction annotation P_{F7} and one backward prediction annotation P_{B7}.

The merger 144 is configured to receive and merge the forward prediction annotation P_{F2} and the backward prediction annotation P_{B2} regarding to the slice image SL2, and generate a final annotation label P₂ on the slice image SL2. Similarly, the merger 144 is also configured to receive and merge the forward prediction annotation P_{F3} and the backward prediction annotation P_{B3} regarding to the slice image SL3, and generate a final annotation label P₃ on the slice image SL3. Similarly, the merger 144 is also configured to receive and merge the forward prediction annotation P_{F7} and the backward prediction annotation P_{B7} regarding to the slice image SL7, and generate a final annotation label P₇ on the slice image SL7.

In some embodiments, the final annotation labels P₂ to P₇ can be determined by the merger 144 according to a distance between a target slice image and the manual-labeled image.

For example, regarding to a K^{th} slice image of the serialized medical images, a final annotation label on the K^{th} slice image is produced by calculating a first distance between the slice image SL1 and the K^{th} slice image with a second distance between the K^{th} slice image and the slice image SL8. If the first distance is shorter than the second distance, the forward prediction annotation on the K^{th} slice image is selected by the merger 144 as the final annotation label on the K^{th} slice image. If the second distance is shorter than the first distance, the backward prediction annotation on the K^{th} slice image is selected by the merger 144 as the final annotation label on the K^{th} slice image. K is a positive integer.

For example, regarding to the slice image SL2, the first distance between the slice image SL1 and the slice image SL2 is “1” and the second distance between the slice image SL2 and the slice image SL8 is “6”. The first distance is shorter. In other words, the slice image SL2 is closer to the slice image SL1. In this case, the forward prediction annotation P_{F2} will be selected by the final annotation label P₂ on the slice image SL2.

Regarding to the slice image SL3, the first distance between the slice image SL1 and the slice image SL3 is “2” and the second distance between the slice image SL3 and the slice image SL8 is “5”. In this case, the forward prediction annotation P_{F3} will be selected by the final annotation label P₃ on the slice image SL3.

Regarding to the slice image SL7, the first distance between the slice image SL1 and the slice image SL7 is “6” and the second distance between the slice image SL7 and the slice image SL8 is “1”. The second distance is shorter. In other words, the slice image SL7 is closer to the slice image SL8. In this case, the backward prediction annotation P_{B7} will be selected by the final annotation label P₇ on the slice image SL7.

In some embodiments, the final annotation labels P₂ to P₇ are not limited to be selected from the forward prediction annotation and the backward prediction annotation. In other embodiments, the final annotation labels P₂ to P₇ can be determined by the merger 144 according to a weighted sum between forward prediction annotation and the backward prediction annotation.

For example, regarding to a K^{th} slice image of the serialized medical images, a final annotation label on the K^{th} slice image is produced by calculating a first distance between the slice image SL1 and the K^{th} slice image with a second distance between the K^{th} slice image and the slice image SL8, and then generating the final annotation label on the K^{th} slice image based on a weighted sum between a forward prediction annotation and a backward prediction annotation on the K^{th} slice image according to the first distance and the second distance.

For example, regarding to the slice image SL2, the first distance between the slice image SL1 and the slice image SL2 is “1” and the second distance between the slice image SL2 and the slice image SL8 is “6”. The final annotation label P₂ on the slice image SL2 can be generated by a weighted sum between the forward prediction annotation P_{F2} and the backward prediction annotation P_{B2}, as below:

$${P}_{2} = \frac{6}{7} {P}_{F 2} + \frac{1}{7} {P}_{B 2}$$

In some embodiments, the forward prediction annotation P_{F2} includes possibility values (e.g., 0% to 100%) on pixels of the slice image SL2. The possibility values represent whether the pixels are relative to the target object or the label. The backward prediction annotation P_{B2} also includes possibility values (e.g., 0% to 100%) on pixels of the slice image SL2. The merger 144 is configured to calculate the weighted sums between the possibility values of the forward prediction annotation P_{F2} and the backward prediction annotation P_{B2}. In some example, if a weighted sum on one pixel is above 50%, this pixel will be selected to be included in the final annotation label P₂ on the slice image SL2. In some example, if a weighted sum on one pixel is below 50%, this pixel will be excluded from the final annotation label P₂ on the slice image SL2.

Regarding to the slice image SL3, the first distance between the slice image SL1 and the slice image SL3 is “2” and the second distance between the slice image SL3 and the slice image SL8 is “5”. The final annotation label P₃ on the slice image SL3 can be generated by a weighted sum between the forward prediction annotation P_{F3} and the backward prediction annotation P_{B3}, as below:

$${P}_{3} = \frac{5}{7} {P}_{F 3} + \frac{2}{7} {P}_{B 3}$$

Regarding to the slice image SL7, the first distance between the slice image SL1 and the slice image SL7 is “6” and the second distance between the slice image SL7 and the slice image SL8 is “1”. The final annotation label P₇ on the slice image SL7 can be generated by a weighted sum between the forward prediction annotation P_{F7} and the backward prediction annotation P_{B7}, as below:

$${P}_{7} = \frac{1}{7} {P}_{F 7} + \frac{6}{7} {P}_{B 7}$$

The bidirectional inference mechanism, which includes the tracking model 142 and the merger 144 shown in FIG. 2, is able to seamlessly integrate with the Track-Anything-Model framework. The medical imaging annotation device 100 can effectively utilize a limited amount of user-annotated data (i.e., the manual annotations P_{M1} and P_{M8}) and generate the final annotation labels P₂ to P₇ on the intermediate slice images automatically according to the bidirectional tracking. The medical imaging annotation device 100 is able to improve semi-automated annotation efficiency. Because the bidirectional inference mechanism provides more information for tracking the object, it enhances the automatic annotation accuracy of accurately identifying unlabeled image data.

The manual annotations P_{M1} and P_{M8} and the final annotation labels P₂ to P₇ on the serialized medical images SIMG can be utilized as training data to train a medical-related model, such an organ segmentation model, a medical imaging classification model, a diagnosis-aiding model.

The bidirectional inference mechanism, which includes the tracking model 142 and the merger 144 shown in FIG. 2, is a post-fusion (decision-level fusion) method. It is simple and easy to implement. In some embodiments, the bidirectional inference mechanism shown in FIG. 2 may result in discontinuous shapes in the generated annotations. Sometimes, it requires further manual adjustments to refine the final annotation labels P₂ to P₇.

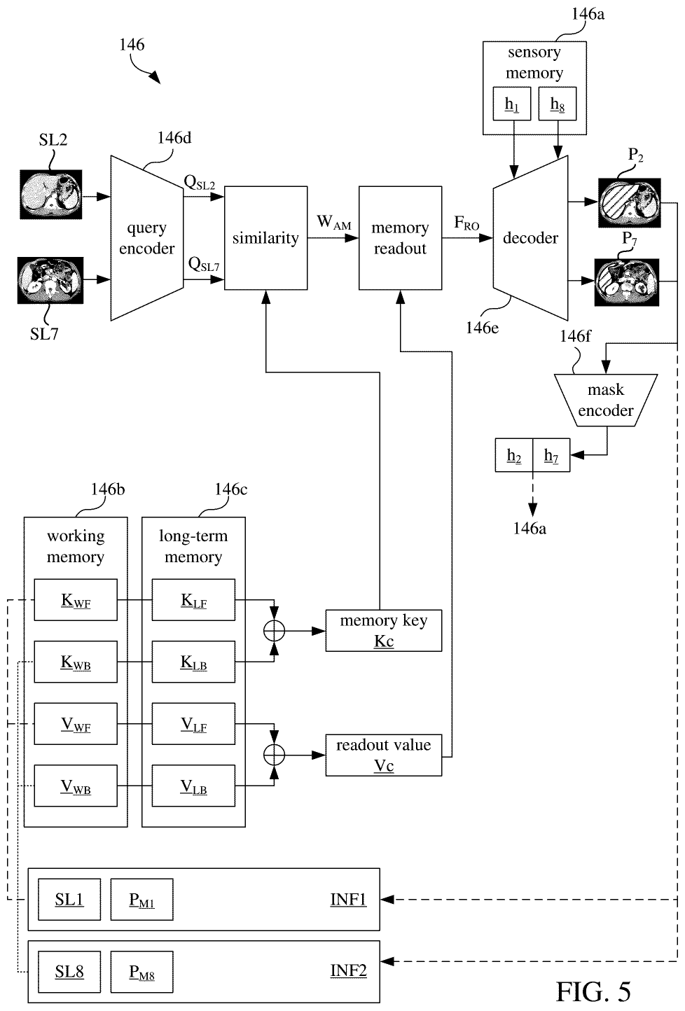

To overcome the potential shape discontinuity issues introduced by Post-Fusion when combining annotation information, the disclosure provide another manner to perform the bidirectional inference mechanism. Reference is further made to FIG. 5, which is a schematic diagram illustrating a bidirectional XMem model 146 executed by the processor 140 according to an embodiment of the disclosure. In some embodiments, the processor 140 is configured to execute the bidirectional XMem model 146 as a manner to implement the bidirectional inference mechanism for generating the final annotation labels P₂, P₃, P₄, P₅, P₆ and P₇ on the intermediate slice images (e.g., the slice images SL2, SL3, SL4, SL5, SL6 and SL7) based on the manual annotations P_{M1} and P_{M8}.

XMem is a model proposed for long-term video object segmentation (VOS), drawing inspiration from the Atkinson-Shiffrin memory model, which consists of short-term and long-term memory systems.

As shown in FIG. 5, the bidirectional XMem model 146 is an improved version of the XMem. The bidirectional XMem model 146 using a bidirectional mechanism is able to consider information from slices both ahead and behind. This bidirectional memory retrieval enhances a contextual understanding of the bidirectional XMem model 146. The bidirectional XMem model 146 adopts a feature-level fusion strategy (not a decision-level fusion). This strategy allows for integration at the feature level, thereby making full use of the model's ability to consider both temporal and spatial dimensions in feature representation. Through feature-level space fusion, annotation information from different perspectives can be combined more closely and effectively, reflecting multi-dimensional analytical capabilities of the bidirectional XMem model 146.

In some embodiments, the bidirectional XMem model 146 is configured to generate the final annotation labels on the intermediate slice images based on the manual annotation P_{M1} and the second manual annotation P_{M8}.

In some embodiments, the bidirectional XMem model 146 is able to process a pair of slice images of the serialized medical images SIMG at one time to generate final annotation labels on the pair of slice images. For example, after receiving the serialized medical images SIMG (including the slice image SL1 to SL8), the manual annotation P_{M1} on the slice image SL1 and the manual annotation P_{M8} on the slice image SL8, the bidirectional XMem model 146 may generate the final annotation labels P₂ and P₇ on one pair of the slice images SL2 and SL7 at first.

As shown in FIG. 5, the bidirectional XMem model 146 includes a sensory memory 146a, a working memory 146b and a long-term memory 146c.

The sensory memory 146a is a short-term memory, which is responsible for handling immediate data from the most recent slices of the serialized medical images SIMG. The sensory memory 146a captures and processes transient information that is crucial for the segmentation of current frames. As shown in FIG. 5, the sensory memory 146a is configured to store a short-term forward hidden representation h₁ and a short-term backward hidden representation h₈. The short-term forward hidden representation h₁ is generated according to the manual annotation P_{M1}. The short-term forward hidden representation h₁ is able to reflect a distribution of the manual annotation P_{M1} (mask/label) on the slice image SL1. The short-term backward hidden representation h₈ is generated according to the manual annotation P_{M8} (mask/label) on the slice image SL8.

The working memory 146b and the long-term memory 146c retain crucial information from previous frames that may no longer be present in the short-term memory. The working memory 146b and the long-term memory 146c are used to maintain context over the serialized medical images SIMG. The working memory 146b can be thought of as a dynamic, flexible storage space that handles information actively and is used for immediate processing tasks. It deals with current and recent frames, allowing the system to adapt quickly to changes in the input. In contrast to the working memory 146b, the long-term memory 146c stores information deemed important for maintaining consistency and context over the serialized medical images SIMG. The long-term memory 146c retains crucial object features and contextual data that persist over longer timescales.

As shown in FIG. 5, the working memory 146b is configured to store a forward memory key feature K_{WF}, a backward memory key feature K_{WB}, a forward memory value feature V_{WF}, a backward memory value feature V_{WB}. The long-term memory 146c is configured to store a forward long-term memory key feature K_{LF}, a backward long-term memory key feature K_{LB}, a forward long-term memory value feature V_{LF}, a backward long-term memory value feature V_{LB}.

In some embodiments, the forward memory key feature K_{WF} and the forward memory value feature V_{WF} in the working memory 146b are updated according to forward information data INF1. At the beginning, the forward information data INF1 include the slice image SL1 and the manual annotation P_{M1}. On the other hand, the backward memory key feature K_{WB} and the backward memory value feature V_{WB} in the working memory 146b are updated according to backward information data INF2. At the beginning, the backward information data INF2 include the slice image SL8 and the manual annotation P_{M8}.

Using a bidirectional mechanism, data stored in the working memory 146b and the long-term memory 146c will considers information (e.g., INF1 and INF2) from slices both ahead and behind. This bidirectional memory retrieval enhances the contextual understanding of the bidirectional XMem model 146.

As shown in FIG. 5, the bidirectional XMem model 146 further includes a query encoder 146d, a decoder 146e and a mask encoder 146f. As shown in FIG. 5, the query encoder 146d is configured to generate a forward input query Q_{SL2} about a forward input medical image (i.e., the slice image SL2) and a backward input query Q_{SL7} about a backward input medical image (i.e., the slice image SL7). The query encoder 146d is responsible for transforming the incoming slice images SL2 and SL7 into high-dimensional feature representations (i.e., the forward input query Q_{SL2} and the backward input query Q_{SL7}). The forward input query Q_{SL2} and the backward input query Q_{SL7} are then used to query the working memory 146b and the long-term memory 146c to retrieve relevant past information.

As shown in FIG. 5, in some embodiments, the forward memory key feature K_{WF}, the backward memory key feature K_{WB}, the forward long-term memory key feature K_{LF} and the backward long-term memory key feature K_{LB} are concatenated to form a memory key Kc. The bidirectional XMem model 146 executed by the processor 140 is configured to calculate the affinity matrix W_{AM} according to a similarity between the forward input query Q_{SL2} and the backward input query Q_{SL7} and the memory key Kc. In other words, the affinity matrix W_{AM} is calculated by the processor 140 according to the forward input query Q_{SL2}, the backward input query Q_{SL7}, the forward memory key feature K_{WF}, the backward memory key feature K_{WB}, the forward long-term memory key feature K_{LF} and the backward long-term memory key feature K_{LB}.

The bidirectional XMem model 146 merges the memories retrieved at different times (mid-term and long-term) to obtain a unified and comprehensive memory representation, such as the memory key Kc. Different from the original XMem, the bidirectional XMem model 146 ensures a more complete representation of shape and temporal information. It is assumed that the shape information from the early, mid, and late stages of serialized images can mutually assist each other, which is particularly useful in medical imaging for tracking objects.

As shown in FIG. 5, the bidirectional XMem model 146 performs a memory readout operation to generate a readout feature F_{RO} according to the affinity matrix W_{AM} and a readout value Vc. In some embodiments, the readout value Vc is calculated by the processor 140 according to the forward memory value feature V_{WF}, the backward memory value feature V_{WB}, the forward long-term memory value feature V_{LF} and the backward long-term memory value feature V_{LB}. The memory readout operation computes the correlation (i.e., the affinity matrix W_{AM}) of the merged memory with the features extracted by the query encoder 146d to identify which features or regions are most relevant to the target object.

The decoder 146e is configured to generate a forward annotation mask (i.e., the final annotation label P₂) about the forward input medical image (i.e., the slice image SL2) and a backward annotation mask (i.e., the final annotation label P₇) about the backward input medical image (i.e., the slice image SL7) according to the forward input query Q_{SL2}, the backward input query Q_{SL7}, the readout feature F_{RO}, the short-term forward hidden representation h₁ and the short-term backward hidden representation h₈ from the sensory memory 146a.

The decoder 146e is to generate the final segmentation masks from the combined features obtained from the query encoder 146d and memory modules (the sensory memory 146a, the working memory 146b and the long-term memory 146c). The decoder 146e takes in the enriched feature representation that combines current slice features from the query encoder 146d with relevant historical data retrieved from the memory modules. This integration ensures that both current observations and past context contribute to the segmentation outcomes (the final annotation label P₂ and the final annotation label P₇).

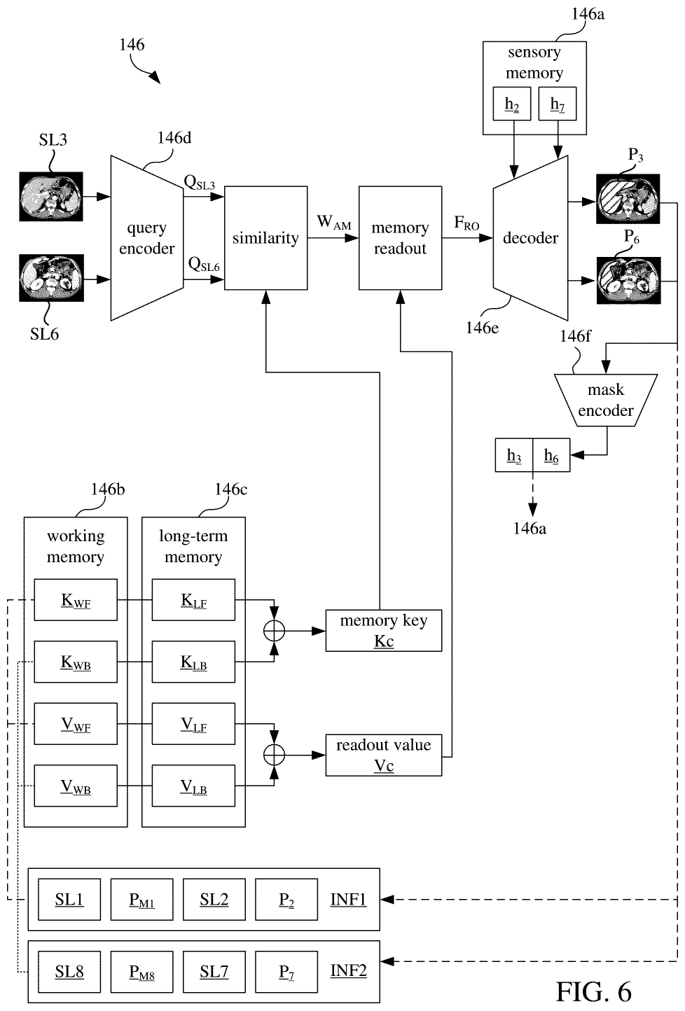

The mask encoder 146f is configured to generate a short-term forward hidden representation h₂ according to the forward annotation mask (i.e., the final annotation label P₂) and a short-term backward hidden representation h₇ according to the backward annotation mask (i.e., the final annotation label P₇). The short-term forward hidden representation h₂ and the short-term backward hidden representation h₇ are updated into the sensory memory 146a for following segmentation regarding to another pair of slice images (will be discussed in FIG. 6).

In this case, the final annotation label P₂ about the slice image SL2 and the final annotation label P₇ about the slice image SL7 can be generated by the bidirectional XMem model 146 in reference with the manual annotation P_{M1} on the slice image SL1 and the manual annotation P_{M8} on the slice image SL8. As shown in FIG. 5, the final annotation label P₂ about the slice image SL2 will be added to the forward information data INF1, and the final annotation label P₇ about the slice image SL7 will be added to the backward information data INF2, for following segmentation.

Reference is further made to FIG. 6, which is a schematic diagram illustrating the bidirectional XMem model 146 executed by the processor 140 regarding to another pair of slice images SL3 and SL6.

As shown in FIG. 6, the sensory memory 146a is configured to store the short-term forward hidden representation h₂ and the short-term backward hidden representation h₇. The short-term forward hidden representation h₂ is able to reflect a distribution of the forward annotation mask (i.e., the final annotation label P₂) on the slice image SL2. The short-term backward hidden representation h₇ is able to reflect a distribution of the backward annotation mask (i.e., the final annotation label P₇) on the slice image SL7.

In some embodiments, the forward memory key feature K_{WF} and the forward memory value feature V_{WF} in the working memory 146b are updated according to forward information data INF1. Currently, the forward information data INF1 include the slice image SL1, the manual annotation P_{M1}, the slice image SL2 and the final annotation label P₂. On the other hand, the backward memory key feature K_{WB} and the backward memory value feature V_{WB} in the working memory 146b are updated according to backward information data INF2. Currently, the backward information data INF2 include the slice image SL8, the manual annotation P_{M8}, the slice image SL7 and the final annotation label P₇.

As shown in FIG. 6, the query encoder 146d is configured to generate a forward input query Q_{SL3} about a forward input medical image (i.e., the slice image SL3) and a backward input query Q_{SL6} about a backward input medical image (i.e., the slice image SL6). The query encoder 146d is responsible for transforming the incoming slice images SL3 and SL6 into high-dimensional feature representations (i.e., the forward input query Q_{SL3} and the backward input query Q_{SL6}).

As shown in FIG. 6, in some embodiments, the forward memory key feature K_{WF}, the backward memory key feature K_{WB}, the forward long-term memory key feature K_{LF} and the backward long-term memory key feature K_{LB} are concatenated to form a memory key Kc. The bidirectional XMem model 146 executed by the processor 140 is configured to calculate the affinity matrix W_{AM} according to a similarity between the forward input query Q_{SL3} and the backward input query Q_{SL6} and the memory key Kc.

As shown in FIG. 6, the bidirectional XMem model 146 performs a memory readout operation to generate a readout feature F_{RO} according to the affinity matrix W_{AM} and the readout value Vc.

The decoder 146e is configured to generate a forward annotation mask (i.e., the final annotation label P₃) about the forward input medical image (i.e., the slice image SL3) and a backward annotation mask (i.e., the final annotation label P₆) about the backward input medical image (i.e., the slice image SL6) according to the forward input query Q_{SL3}, the backward input query Q_{SL6}, the readout feature F_{RO}, the short-term forward hidden representation h₂ and the short-term backward hidden representation h₇ from the sensory memory 146a.

The decoder 146e is to generate the final segmentation masks from the combined features obtained from the query encoder 146d and memory modules (the sensory memory 146a, the working memory 146b and the long-term memory 146c). The decoder 146e takes in the enriched feature representation that combines current slice features from the query encoder 146d with relevant historical data retrieved from the memory modules. This integration ensures that both current observations and past context contribute to the segmentation outcomes (the final annotation label P₂ and the final annotation label P₇).

The mask encoder 146f is configured to generate a short-term forward hidden representation h₃ according to the forward annotation mask (i.e., the final annotation label P₃) and a short-term backward hidden representation h₆ according to the backward annotation mask (i.e., the final annotation label P₆). The short-term forward hidden representation h₃ and the short-term backward hidden representation h₆ are updated into the sensory memory 146a for following segmentation regarding to another pair of slice images.

In this case, the final annotation label P₃ about the slice image SL3 and the final annotation label P₆ about the slice image SL6 can be generated by the bidirectional XMem model 146 in reference with current input data and historical data. As shown in FIG. 6, the final annotation label P₃ about the slice image SL3 will be added to the forward information data INF1, and the final annotation label P₆ about the slice image SL6 will be added to the backward information data INF2, for following segmentation.

Similarly, final annotation labels P₄ and P₅ regarding to other slice images SL4 and SL5 can be generated by the bidirectional XMem model 146.

In some embodiments, the query encoder 146d, the decoder 146e and the mask encoder 146f shown in FIG. 5 and FIG. 6 can be implemented by software instructions executed by the processor 140 shown in FIG. 1. In some embodiments, the sensory memory 146a, the working memory 146b and the long-term memory 146c shown in FIG. 5 and FIG. 6 can be implemented by memory regions defined in the memory 160 shown in FIG. 1 or implemented individual memory components.

As shown in aforementioned embodiments, the medical imaging annotation device 100 can handle various kinds of continuous data such as serialized images and medical videos simultaneously. It effectively leverages multiple different annotation data using the bidirectional inference mechanism.

In some embodiments, the medical imaging annotation device 100 can be implemented by a computer, a computing server or a medical image server. The processor 140 can be implemented by a central processing unit, a graphic processing unit, a tensor processing unit or an ASIC.

A medical imaging annotation method executed by the medical imaging annotation device 100 is also an embodiment of this disclosure. The medical imaging annotation method include steps of: obtaining the serialized medical images SIMG; receiving a first manual annotation (e.g., the manual annotation P_{M1}) on a first slice image (e.g., the slice image SL1) of the serialized medical images SIMG and a second manual annotation (e.g., the manual annotation P_{M8}) on a second slice image (e.g., the slice image SL8) of the serialized medical images SIMG; executing a bidirectional inference mechanism (e.g., the tracking model 142 and the merger 144 shown in FIG. 2, or the bidirectional XMem model 146 shown in FIG. 5 and FIG. 6) to generate final annotation labels (e.g., the final annotation labels P₂ to P₇) on intermediate slice images (e.g., the slice images SL2 to SL7) of the serialized medical images SIMG based on the first manual annotation (e.g., the manual annotation P_{M1}) and the second manual annotation (e.g., the manual annotation P_{M8}) respectively. Details about these steps are already discussed in aforesaid embodiments, and not repeated again.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A medical imaging annotation device, comprising:
an interface;
a memory, storing serialized medical images; and
a processor, coupled to the interface and the memory, and the processor being configured to:
receive, via the interface, a first manual annotation on a first slice image of the serialized medical images and a second manual annotation on a second slice image of the serialized medical images; and
execute a bidirectional inference mechanism to generate final annotation labels on intermediate slice images of the serialized medical images based on the first manual annotation and the second manual annotation respectively.

2. The medical imaging annotation device of claim 1, wherein the bidirectional inference mechanism executed by the processor comprises:
executing a tracking model based on the first manual annotation on the first slice image, for generating forward prediction annotations on the intermediate slice images of the serialized medical images sequentially in a forward order;
executing the tracking model based on the second manual annotation on the second slice image, for generating backward prediction annotations on the intermediate slice images of the serialized medical images sequentially in a backward order; and
merging the forward prediction annotations and the backward prediction annotations to produce the final annotation labels on the intermediate slice images.

3. The medical imaging annotation device of claim 2, wherein the tracking model comprises a Track-Anything-Model (TAM), the Track-Anything-Model is configured to detect, associate, and follow the first manual annotation on the first slice image across the intermediate slice images in the forward order, and the Track-Anything-Model is configured to detect, associate, and follow the second manual annotation on the second slice image across the intermediate slice images in the backward order.

4. The medical imaging annotation device of claim 2, wherein regarding to a K^{th} slice image of the serialized medical images, a final annotation label on the K^{th} slice image is produced by:
calculating a first distance between the first slice image and the K^{th} slice image with a second distance between the K^{th} slice image and the second slice image;
in response to the first distance is shorter than the second distance, selecting a forward prediction annotation on the K^{th} slice image as the final annotation label on the K^{th} slice image; and
in response to the second distance is shorter than the first distance, selecting a backward prediction annotation on the K^{th} slice image as the final annotation label on the K^{th} slice image.

5. The medical imaging annotation device of claim 2, wherein regarding to a K^{th} slice image of the serialized medical images, a final annotation label on the K^{th} slice image is produced by:
calculating a first distance between the first slice image and the K^{th} slice image with a second distance between the K^{th} slice image and the second slice image; and
generating the final annotation label on the K^{th} slice image based on a weighted sum between a forward prediction annotation and a backward prediction annotation on the K^{th} slice image according to the first distance and the second distance.

6. The medical imaging annotation device of claim 1, wherein the bidirectional inference mechanism executed by the processor comprises:
executing a bidirectional XMem model for generating the final annotation labels on the intermediate slice images based on the first manual annotation and the second manual annotation.

7. The medical imaging annotation device of claim 6, wherein the bidirectional XMem model comprises:
a sensory memory, configured to store a short-term forward hidden representation and a short-term backward hidden representation;
a working memory, configured to store a forward memory key feature, a backward memory key feature, a forward memory value feature, a backward memory value feature; and
a long-term memory, configured to store a forward long-term memory key feature, a backward long-term memory key feature, a forward long-term memory value feature, a backward long-term memory value feature.

8. The medical imaging annotation device of claim 7, wherein the bidirectional XMem model further comprises:
a query encoder, configured to generate a forward input query about a forward input medical image and a backward input query about a backward input medical image;
a decoder, configured to generate a forward annotation mask about the forward input medical image and a backward annotation mask about the backward input medical image according to the forward input query, the backward input query, a readout feature, the short-term forward hidden representation and the short-term backward hidden representation from the sensory memory; and
a mask encoder, configured to generate the short-term forward hidden representation and the short-term backward hidden representation according to the forward annotation mask and the backward annotation mask.

9. The medical imaging annotation device of claim 8, wherein the readout feature is generated according to an affinity matrix and a readout value,
the affinity matrix is calculated by the processor according to the forward input query, the backward input query, the forward memory key feature, the backward memory key feature, the forward long-term memory key feature and the backward long-term memory key feature,
the readout value is calculated by the processor according to the forward memory value feature, the backward memory value feature, the forward long-term memory value feature and the backward long-term memory value feature.

10. The medical imaging annotation device of claim 1, wherein the serialized medical images comprises Magnetic Resonance Imaging (MRI) scan images or Computed Tomography (CT) scan images.

11. A medical imaging annotation method, comprising:
obtaining serialized medical images;
receiving a first manual annotation on a first slice image of the serialized medical images and a second manual annotation on a second slice image of the serialized medical images; and
execute a bidirectional inference mechanism to generate final annotation labels on intermediate slice images of the serialized medical images based on the first manual annotation and the second manual annotation respectively.

12. The medical imaging annotation method of claim 11, wherein the bidirectional inference mechanism comprises:
executing a tracking model based on the first manual annotation on the first slice image, for generating forward prediction annotations on the intermediate slice images of the serialized medical images sequentially in a forward order;
executing the tracking model based on the second manual annotation on the second slice image, for generating backward prediction annotations on the intermediate slice images of the serialized medical images sequentially in a backward order; and
merging the forward prediction annotations and the backward prediction annotations to produce the final annotation labels on the intermediate slice images.

13. The medical imaging annotation method of claim 12, wherein the tracking model comprises a Track-Anything-Model (TAM), the Track-Anything-Model is configured to detect, associate, and follow the first manual annotation on the first slice image across the intermediate slice images in the forward order, and the Track-Anything-Model is configured to detect, associate, and follow the second manual annotation on the second slice image across the intermediate slice images in the backward order.

14. The medical imaging annotation method of claim 12, wherein regarding to a K^{th} slice image of the serialized medical images, a final annotation label on the K^{th} slice image is produced by:
calculating a first distance between the first slice image and the K^{th} slice image with a second distance between the K^{th} slice image and the second slice image;
in response to the first distance is shorter than the second distance, selecting a forward prediction annotation on the K^{th} slice image as the final annotation label on the K^{th} slice image; and
in response to the second distance is shorter than the first distance, selecting a backward prediction annotation on the K^{th} slice image as the final annotation label on the K^{th} slice image.

15. The medical imaging annotation method of claim 12, wherein regarding to a K^{th} slice image of the serialized medical images, a final annotation label on the K^{th} slice image is produced by:
calculating a first distance between the first slice image and the K^{th} slice image with a second distance between the K^{th} slice image and the second slice image; and
generating the final annotation label on the K^{th} slice image based on a weighted sum between a forward prediction annotation and a backward prediction annotation on the K^{th} slice image according to the first distance and the second distance.

16. The medical imaging annotation method of claim 11, wherein the bidirectional inference mechanism comprises:
executing a bidirectional XMem model for generating the final annotation labels on the intermediate slice images based on the first manual annotation and the second manual annotation.

17. The medical imaging annotation method of claim 16, wherein executing the bidirectional XMem model comprises:
storing a short-term forward hidden representation and a short-term backward hidden representation in a sensory memory;
storing a forward memory key feature, a backward memory key feature, a forward memory value feature, a backward memory value feature in a working memory; and
storing a forward long-term memory key feature, a backward long-term memory key feature, a forward long-term memory value feature, a backward long-term memory value feature in a long-term memory.

18. The medical imaging annotation method of claim 17, wherein executing the bidirectional XMem model further comprises:
generating, by a query encoder, a forward input query about a forward input medical image and a backward input query about a backward input medical image;
generating, by a decoder, a forward annotation mask about the forward input medical image and a backward annotation mask about the backward input medical image according to the forward input query, the backward input query, a readout feature, the short-term forward hidden representation and the short-term backward hidden representation from the sensory memory; and
generating, by a mask encoder, the short-term forward hidden representation and the short-term backward hidden representation according to the forward annotation mask and the backward annotation mask.

19. The medical imaging annotation method of claim 18, wherein the readout feature is generated according to an affinity matrix and a readout value,
the affinity matrix is calculated according to the forward input query, the backward input query, the forward memory key feature, the backward memory key feature, the forward long-term memory key feature and the backward long-term memory key feature,
the readout value is calculated according to the forward memory value feature, the backward memory value feature, the forward long-term memory value feature and the backward long-term memory value feature.

20. The medical imaging annotation method of claim 11, wherein the serialized medical images comprises Magnetic Resonance Imaging (MRI) scan images or Computed Tomography (CT) scan images.
